(19) United States
(12) Patent Application Publication
(10) Pub. No.: US 2009/0326280 A1
(43) Pub. Date: Dec. 31, 2009

(54) Systems and Methods for the Preparation of Alkyl Aryl Ethers

(76) Inventor: Todd Coleman, Batesville, AR (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 12/492,936

(22) Filed: Jun. 26, 2009

Related U.S. Application Data
(60) Provisional application No. 61/076,214, filed on Jun. 27, 2008.

(51) Int. Cl. C07C 43/225; C07C 41/01

(52) U.S. Cl. 568/656

(57) ABSTRACT

A process for the preparation of alkyl aryl ethers from alcohols and aryl halides, usually as intermediates in organic synthesis. In a method, the mixing aryl halide and an alcohol is mixed with dimethyl sulfoxide, water, and a metal hydroxide to form a mixture and the mixture is heated to reflux. Additional steps may then be performed to provide for purification.

### CROSS REFERENCE TO RELATED APPLICATION(S)

This application claims benefit of U.S. Provisional Patent Application Ser. No. 61/076,214 filed Jun. 27, 2008, the entire disclosure of which is herein incorporated by reference.

### BACKGROUND

1. Field of the Invention

This invention relates to the field of creating alkyl aryl ethers from alcohols and aryl halides. In particular, to the field of creating alkyl aryl ethers where the alcohols and aryl halides are intermediates in the organic synthesis.

2. Description of the Related Art

Ethers are a class of organic compounds which contain an ether group—an oxygen atom connected to two (substituted) alkyl or aryl groups—of general formula R—O—R′.

Ether molecules cannot form hydrogen bonds amongst each other, resulting in a relatively low boiling point compared to that of the analogous alcohols. However, the differences in the boiling points of the ethers and their isometric alcohols become smaller as the carbon chains become longer, as the hydrophobic nature of the carbon chain becomes more predominant over the presence of hydrogen bonding.

Generally, ethers can be prepared in the laboratory in several different ways, such as: intermolecular dehydration of alcohols, nucleophilic displacement of alkyl halides by alkoxides; nucleophilic displacement of alkyl halides by phenoxides, and electrophilic addition of alcohols to alkenes.

Processes for the formation of alkyl aryl ethers from aryl halides are well established. The earliest reference to the process was by Lulofs in 1901 (See Rec. Tav. Chim. 20, 292, 1901). Follow-up work was reported by DeMooy (See Rec. Tav. Chim. 35, 5, 1915) and Holleman (See Rec. Tav. Chim. 37, 195, 1918). In particular, Holleman's procedure used the base in methanol to prepare alkyl aryl ethers from trisubstituted chlorobenzenes.

In still later publications, Rubin and co-workers reported kinetic studies on the formation of 3,5-dichlorophenetole prepared from 1,3,5-trichlorobenzene and sodium ethoxide. Sodium ethoxide was also later generated from metallic sodium and anhydrous ethanol (See J. Am. Chem. Soc. 1953, 75, 2517). Reaction temperatures in these processes were reported to be between 150 and 185° C.

In a still later publication, Ogata and co-workers reported the transetherification of alkyl dinitro substituted aryl ethers (See J. Am. Chem. Soc. 1949, 71, 3211). The reaction in this process was catalyzed by potassium hydroxide. In his manuscript, Ogata clearly states that dinitro substitution is required for the transetherification to proceed. The dihalo substituted starting materials in this procedure were found to be unreactive.

In a still later publication, Sam and co-workers reported on the formation of 2-chloroanisole from 1,2-dichlorobenzene by use of a potassium methoxide crown ether complex (See J. Am. Chem. Soc. 1974, 96, 2252). The paper reports however that in the absence of the crown ether complex, the reaction does not occur.

Later on, Shaw and co-workers reported the preparation of 2-chloroanisole from 1,2-chlorobenzene by use of solid sodium methoxide in hexamethylphosphoramide (See J. Org. Chem. 1976, 41, 732). While around the same time, Bradshaw and co-workers described the use of dimethyl sulfoxide as the solvent for treating monohalonaphthalenes with potassium t-butoxide in t-butanol (See J. Org. Chem. 1971, 36, 314). This procedure formed a complex mixture of ten different reaction products.

One of the more recently established procedures for the formation of alkyl aryl ethers from aryl halides is U.S. Pat. No. 4,454,355, which teaches the preparation of 4-nitrophenetole by the use of ethanol, 50% caustic, and a phase-transfer catalyst.

Each of these known processes, however, has several problems and disadvantages, particularly in manufacturing settings. First, there is a safety issue associated with these known processes, as their use of sodium or potassium metal creates potentially dangerous quantities of hydrogen gas. As hydrogen gas is extremely flammable, this creates a significant safety risk in manufacturing settings. Second, metal alkoxides derived from higher alcohols used in the processes of some of the references can be expensive and are not readily available for commercial purpose. The problems associated with cost and the procurement of a vital raw material can drastically increase costs for both the process employed and the end product.

### SUMMARY

The following is a summary of the invention in order to provide a basic understanding of some aspects of the invention. This summary is not intended to identify key or critical elements of the invention or to delineate the scope of the invention. The sole purpose of this section is to present some concepts of the invention in a simplified form as a prelude to the more detailed description that is presented later.

Because of these and other problems in the art, described herein are, among other things, a method for forming an alkyl aryl ether from aryl halide, the method comprising: providing an aryl halide and an alcohol; mixing said aryl halide and said alcohol with dimethyl sulfoxide, water, and a metal hydroxide to form a mixture; heating said mixture to reflux; and segregating said formed alkyl aryl ether from said mixture.

In an embodiment of the method said aryl halide comprises a halobenzene, such as, but not limited to 1,3,5 Trichlorobenzene and said alcohol may be, but not limited to, 1-propanol or methanol.

In an embodiment of the method said metal hydroxide comprises potassium hydroxide.

In an embodiment of the method said segregating comprises dissolving precipitated salts.

In an embodiment, the method further comprises: adding a hydrocarbon solvent, such as, but not limited to toluene, after to said heating to extract additional alkyl aryl ether from said mixture.

There is also described herein an alkyl aryl ether formed by the process of: providing an aryl halide and an alcohol; mixing said aryl halide and said alcohol with dimethyl sulfoxide, water, and a metal hydroxide to form a mixture; heating said mixture to reflux; and segregating said formed alkyl aryl ether from said mixture.

In an embodiment of forming the alkyl aryl ether said aryl halide comprises a halobenzene, such as, but not limited to 1,3,5 Trichlorobenzene and said alcohol may be, but not limited to, 1-propanol or methanol.

In an embodiment of forming the alkyl aryl ether said metal hydroxide comprises potassium hydroxide.

In an embodiment of forming the alkyl aryl ether said segregating comprises dissolving precipitated salts.

In an embodiment, the forming of the alkyl aryl ether further comprises: adding a hydrocarbon solvent, such as, but not limited to toluene, after to said heating to extract additional alkyl aryl ether from said mixture.

### BRIEF DESCRIPTION OF THE DRAWINGS

FIG. 1A provides for the chemical structures of some alkyl aryl ethers which can be derived using the processes discussed herein. In these X will generally be Chlorine (Cl) or Bromine (Br) and R will be an alkyl, carbocycle, or aryl.

FIG. 1B provides for some specific alkyl aryl ethers which can be derived using the processes discussed herein.

FIG. 2 provides an embodiment of a molecular diagram of a process for the preparation of 3,5-Dichloropropoxybenzene from 1-propanol and 1,3,5-Trichlorobenzene.

FIG. 3 provides a flowchart illustrating an embodiment of a process for the preparation of alkyl aryl ethers.

### DESCRIPTION OF THE PREFERRED EMBODIMENT(S)

The following detailed description illustrates by way of example and not by way of limitation. Described herein, among other things, is a new process for the preparation of alkyl aryl ethers from alcohols and aryl halides, usually as intermediates in organic synthesis.

In an embodiment, this process comprises: using a solvent system of dimethyl sulfoxide and the alcohol of choice to which is added a metal hydroxide base and aryl halide along with an amount of water required to dissolve the metal hydroxide, and heating the mixture to reflux for a determined period of time.

Additional steps may be performed in certain embodiments to provide for purification and improved end product recovery including: removing excess alcohol solvent; isolating the product by the addition of water to dissolve the precipitated salts; use of a strong acid to neutralize remaining base; recovering additional product by extracting the aqueous phase with a hydrocarbon solvent; and cooling and clarifying the distillation residue through a filter to remove trace amounts of insoluble salts.

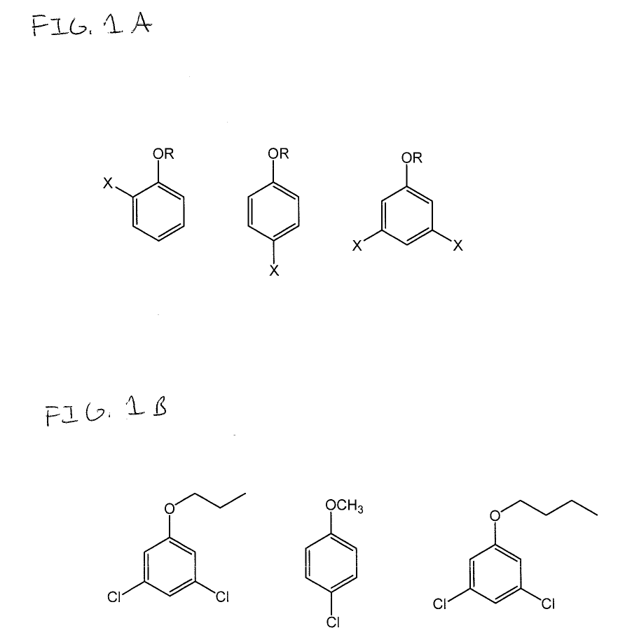

Any type of source alcohol known to those of skill in the art can be used in the disclosed process to produce the desired end product. 1-propanol, methanol, ethanol, butanol, or any other alcohol known to those of skill in the art can be utilized in the processes discussed herein depending on the desired resultant product. Similarly, any initial aryl halide may be selected. Thus the process can be used to produce any desired alkyl aryl ether. Some examples of alkyl aryl ethers which can be produced in accordance with the processes discussed herein are provided in FIGS. 1A and 1B where FIG. 1A provides for exemplary general forms of alkyl aryl ethers that may be performed with X generally being Chlorine (Cl) or Bromine (Br) and R being an alkyl, carbocycle, or aryl. FIG. 1B provides for some specific examples which may be produced and are generally preferred products to be produced by the described systems and methods.

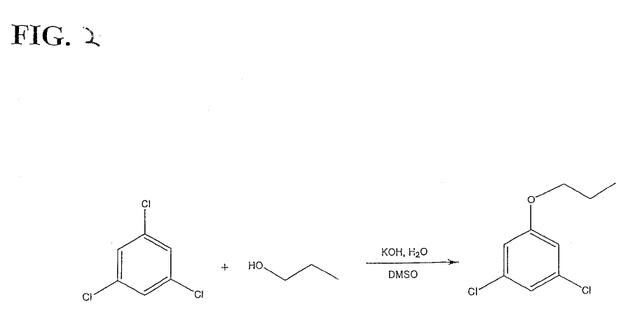

To provide for the operation of the process, one exemplary reaction is provided in molecular diagram in FIG. 2. In this embodiment, the alcohol is 1-propanol which is reacted with the aryl halide 1,3,5-Trichlorobenzene utilizing a mixture of dimethyl sulfoxide (DMSO), water, and potassium hydroxide (KOH) to produce the alkyl aryl ether 3,5-Dichloropropoxybenzene.

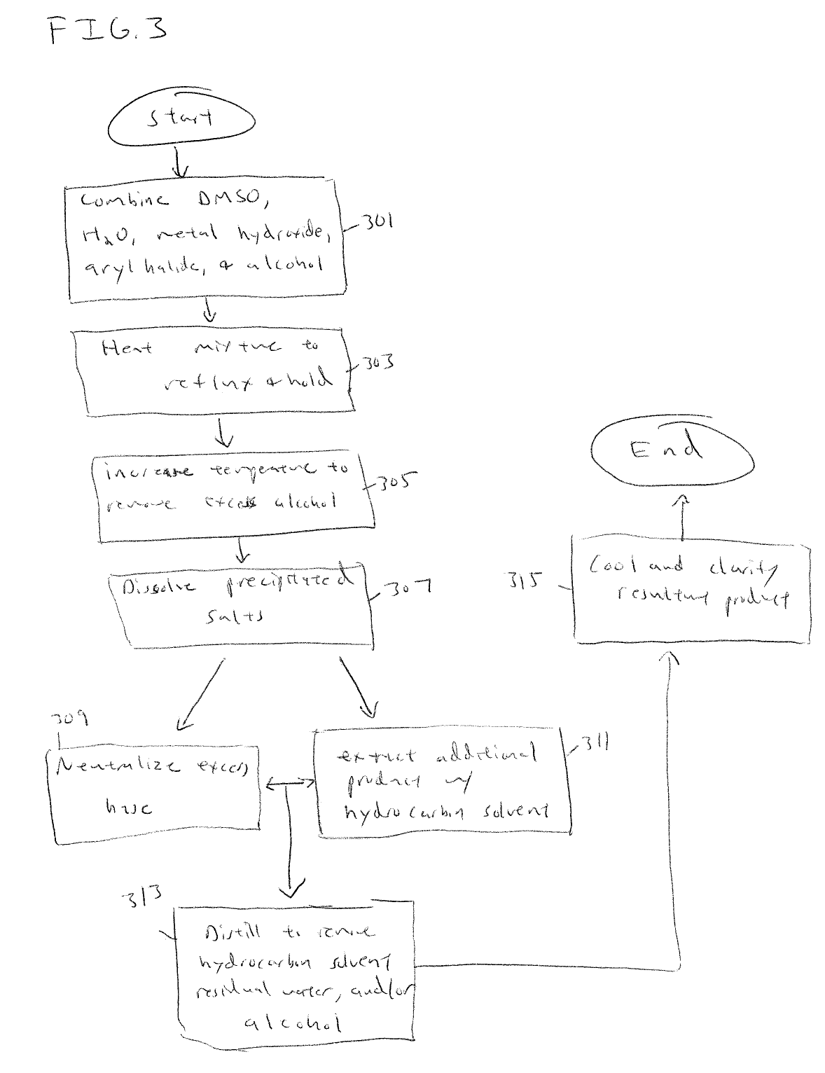

FIG. 3 provides a flowchart of the steps which may be performed to carry out the reaction. In step (301) dimethyl sulfoxide, water, a metal hydroxide and the selected aryl halide (a halobenzene in some embodiments) and alcohol are combined. The mixture is then heated to reflux and held at that heat for a period of time to allow for the reaction to complete (303). The temperature is then increased to distill and remove excess alcohol (305). Precipitated salts may then be dissolved through the addition of water (307) and any additional base may be neutralized (309).

To obtain additional product the aqueous phase may be extracted using a hydrocarbon solvent (311). In an alternative embodiment, the neutralization (309) may alternatively occur after the extraction (311). The extraction mixture is again distilled for removal of the hydrocarbon solvent (313). Finally, the product is then cooled and clarified (315).

If hydrocarbon solvent extraction (311) was not utilized, the latter distillation step (313) would simply be to remove residual water and alcohol. Further, if a higher boiling alcohol was originally used, the product may be crystalline and thus can be isolated by crystallization and filtration in (313) as opposed to distillation in (313) in another embodiment.

For the purpose of this disclosure, it is noted that distillation and volume conditions discussed in the below example are not determinative, and any functional distillation or volume condition known to those of skill in the art is contemplated the process of this disclosure. Generally, reaction temperatures will depend on the initial alcohol chosen and therefore the temperatures disclosed in the embodiments discussed herein are not rigid but will vary in a manner understood by one of ordinary skill in the art depending on the boiling point of the particular alcohol being utilized.

For example, for 1-propanol the reflux temperature is generally about 113-118° C. and this temperature will be used in step (303). However, it will be recognized by one of ordinary skill that the preferred reaction temperature range is a function of the choice of alcohol and higher or lower boiling alcohols may not reach reflux in the above range. If such other alcohols are used, the range will generally be selected so as to produce reflux for that alcohol. For example, if methanol was selected, the reflux temperature, in an embodiment, would generally be about 70-75° C. for step (303). Generally, the maximum temperature to which the mixture should be heated in step (303) is 150° C.

Similarly, the temperature range selected to distil out excess alcohol in step (305) is also dependent on the specific alcohol selected. The upper temperature limit for step (305) is again about 150° C. However, if a lower boiling alcohol is chosen, the temperature range of step (305) may include any value above the boiling point of that alcohol which is still considered to be safe and which does not damage any resulting end products.

A believed advantage of this process, as compared to the other methods discussed previously, is that it offers the ability to generate metal alkoxides in situ, in the presence of water, without the generation of hydrogen gas. The in situ generation of metal alkoxides allows for the use of higher alcohols, typically greater than C₂ as the need to provide expensive metal alkoxides as a reaction precursor is eliminated making the process much more cost effective. An additional advantage is that formation of large amounts of phenols is generally avoided, even in the presence of water.

Elimination of or the reduction in large amounts of hydrogen generally provides for an industrially safer process as it can significantly reduce the risk of fire or explosion due to burning hydrogen gas. Other advantages are that dimethyl sulfoxide is used, which is a preferred solvent over Hexamethylphosphoramide (HMPA) due to its lower toxicity and more ready commercial availability. In addition, no phase-transfer catalyst or crown ether is required as previously described in the literature further simplifying the process.

### Example

The invention now will be described with respect to the following example; however, the scope of the present invention is not intended to be limited thereby.

This example provides for a reaction which may be used to produce 3,5-Dichloropropoxybenzene from 1-propanol and 1,3,5-Trichlorobenzene using dimethyl sulfoxide, water, and potassium hydroxide. Table 1 provides for the materials and amounts used in this example.

TABLE 1

| | Molecular | Assay, | Weight | |
|---|---|---|---|---|
| Reagent | Weight | % | (grams) | Moles |
| 1,3,5- | 181.5 | >99.5 | 100 | 0.55 |
| Trichlorobenzene | | | | |
| 1-Propanol | 60 | 100 | 165 | 2.75 |
| DMSO | 78 | 100 | 100 | |
| 85% KOH | 56 | 85 | 45 | 0.68 |
| Water (Reaction) | 18 | 100 | 10 | |
| Water (Work-up) | 18 | 100 | 200 | |
| 36% HCl | 36 | 36 | 15 | |
| Toluene | 92 | 100 | 87 | |

To begin, a 500-mL round bottom flask with bottom stopcock was charged with the 100 grams Dimethyl Sulfoxide and agitation was begun.

The potassium hydroxide base and 1 propanol was then added to the solvent along with an amount of water to dissolve the potassium hydroxide. The mixture was heated to 55-60° C. and agitated until all the potassium hydroxide had dissolved.

The 1,3,5-Trichlorobenzene was then added to the mixture and the mixture was heated to reflux (generally about 113-118° C. for 1-propanol) and held at that temperature for 3 hours.

Excess alcohol solvent was then removed via distillation comprising increasing temperature to generally about 130-135° C. for 1-propanol and distillate was collected over 3 hours.

The product was then isolated through the addition of water to dissolve the precipitated salts by charging 200-ml of water and agitating for 15 minutes at 50-60° C. In an embodiment, a strong acid may additionally be added in an embodiment to neutralize the remaining base at this point. Alternatively this step can be performed after the toluene extraction. In either case, the neutralization is generally performed by charging the flask with HCl.

Agitation was stopped and the batch is settled for 30 minutes while maintaining the temperature at 50-60° C. The bottom product layer from the flask was decanted and the weight of the bottom product layer was recorded.

In order to recover additional product by extracting the aqueous phase with a hydrocarbon solvent, 87.0 grams toluene was charged to the flask while maintaining the temperature at 50-60° C. and agitating. The agitation was stopped and the batch was settled for 30 minutes while maintaining the temperature at 50-60° C. The bottom aqueous waste layer was drained and discarded appropriately.

The hydrocarbon extract and the initial product phase were combined and vacuum distilled reducing pressure to 50-70 mm Hg, and slowly increasing the pot temperature to 130° C. to remove low boilers.

The distillation residue was run through a paper filter to remove residual insolubles into a suitable container and reduced to room temperature. The process resulted in an output of 3,5-Dichloropropoxybenzene.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A method for forming an alkyl aryl ether from aryl halide, the method comprising:
providing an aryl halide and an alcohol;
mixing said aryl halide and said alcohol with dimethyl sulfoxide, water, and a metal hydroxide to form a mixture;
heating said mixture to reflux; and
segregating said formed alkyl aryl ether from said mixture.

2. The method of claim 1 wherein said aryl halide comprises a halobenzene.

3. The method of claim 2 wherein said halobenzene comprises 1,3,5 Trichlorobenzene.

4. The method of claim 2 wherein said alcohol comprises 1-propanol.

5. The method of claim 1 wherein said alcohol comprises 1-propanol.

6. The method of claim 1 wherein said metal hydroxide comprises potassium hydroxide.

7. The method of claim 1 wherein said segregating comprises dissolving precipitated salts;

8. The method of claim 1 further comprising:
adding a hydrocarbon solvent after to said heating to extract additional alkyl aryl ether from said mixture.

9. The method of claim 8 wherein said hydrocarbon solvent comprises toluene.

10. The method of claim 1 wherein said alcohol comprises methanol.

11. An alkyl aryl ether formed by the process of:
providing an aryl halide and an alcohol;
mixing said aryl halide and said alcohol with dimethyl sulfoxide, water, and a metal hydroxide to form a mixture;
heating said mixture to reflux; and
segregating said formed alkyl aryl ether from said mixture.

12. The product of claim 1 wherein said aryl halide comprises a halobenzene.

13. The product of claim 2 wherein said halobenzene comprises 1,3,5 Trichlorobenzene.

14. The product of claim 2 wherein said alcohol comprises 1-propanol.

15. The product of claim 1 wherein said alcohol comprises 1-propanol.

16. The product of claim 1 wherein said metal hydroxide comprises potassium hydroxide.

17. The product of claim 1 wherein said segregating comprises dissolving precipitated salts;

18. The product of claim 1 further comprising:
adding a hydrocarbon solvent after to said heating to extract additional alkyl aryl ether from said mixture.

19. The product of claim 8 wherein said hydrocarbon solvent comprises toluene.

20. The method of claim 11 wherein said alcohol comprises methanol.
